(19) United States
(12) Patent Application Publication
(10) Pub. No.: US 2013/0096670 A1
(43) Pub. Date: Apr. 18, 2013

(54) Expandable Medical Implant

(71) Applicants: Wolfgang Goetz, Regensburg (DE); Hou-Sen Lim, Singapore (SG)

(72) Inventors: Wolfgang Goetz, Regensburg (DE); Hou-Sen Lim, Singapore (SG)

(73) Assignee: TRANSCATHETER TECHNOLOGIES GMBH, Regensburg (DE)

(21) Appl. No.: 13/579,917

(22) PCT Filed: Feb. 16, 2011
(86) PCT No.: PCT/EP2011/000735

§ 371 Date: Dec. 27, 2012

Related U.S. Application Data
(60) Provisional application No. 61/306,564, filed on Feb. 22, 2010.

(30) Foreign Application Priority Data
Feb. 17, 2011 (DE) 102010008362.3

(51) Int. Cl. A61F 2/82

(52) U.S. Cl. CPC A61F 2/82
USPC 623/1.16

(57) ABSTRACT

An implant includes at least one supporting arrangement which is suited for supporting the implant at or on an implantation position, wherein both the supporting arrangement and the implant are expandable from a first diameter to a second diameter and/or are collapsible from the second diameter to the first diameter. The supporting arrangement includes bars which are connected to each other by means of connecting sections and at least one post for connecting the supporting arrangement with at least one other structure of the implant. At least two of the connecting sections differ in at least one material characteristic, for example, thickness.

The present invention relates to a medical implant having the features of claim 1 and/or 8. It further relates to a set having the features of claim 11 and to a supporting means having the features of claim 12.

Medical implants such as stents, implantable cardiac valve supporting devices, and the like, are usually folded or collapsed, respectively, before they are inserted into the patient's body and are then inserted, e.g., shifted, to the implantation site by means of suitable tools. The unfolding of the folded or collapsed implant is performed at the implantation site, either actively or passively.

Thereby, it can be necessary to re-fold or re-collapse, respectively, an implant which has been placed incorrectly in the target organ (in a vessel or another organ, see above) or to reduce the diameter of the implant in order to re-implant it at or in the originally intended site.

An object of the present invention is to propose a suitable design or embodiment of the expandable medical implant.

This object according to the present invention is solved by the feature combination according to claim 1 and also solved by the feature combination according to claim 8.

Thus, according to the invention, there is proposed an expandable and/or decollapsible implant, in particular a stent, comprising at least one supporting means which is suited for supporting the implant at or on an implantation site. Both the supporting means and the implant are expandable from a respective first diameter to a respective second diameter and/or are collapsible from the second diameter to the first diameter. The supporting means comprises bars which are connected to each other by means of connecting sections; and at least one post for connecting the supporting means with at least one other structure of the implant. Thereby, at least two of the connecting sections differ in at least one material characteristic.

Advantageous embodiments and developments of the present invention are subject-matter of the dependent claims.

The at least one material characteristic in which at least two of the connecting sections differ from another is, in some of the embodiments according to the invention, understood as a thickness of the respective connecting sections.

In some of the embodiments according to the invention, the material characteristic is understood such that the connecting sections differing therein are different from another as regards the selection of their manufacturing material, i.e. chemically.

In some of the embodiments according to the invention, the material characteristic is understood as a combination of a different thickness in an arbitrary direction and of a different material selection.

In some of the embodiments according to the invention, the material characteristic refers to a thickness of at least one section of the connecting sections in a longitudinal direction of the implant and/or in a direction present at a right angle thereto (lateral direction). In an implant which is perfused by bodily fluids after its implantation, the longitudinal direction of the implant can correspond to the direction of the (main) perfusion. The lateral direction of the implant can be a direction in which a (main) expansion of the supporting means takes place (e.g., at the implantation site in the patient's body or before the implantation in a laboratory without any external application of force to the supporting means).

In some of the embodiments of the implant according to the invention, a first connecting section having the smallest distance to a post has a first thickness d1. Thereby, this thickness is the smallest thickness of all thicknesses of the connecting sections.

In some of the embodiments of the implant according to the invention, a second connecting section has a second thickness d2. Thickness d2 is larger than thickness d1. In some embodiments, the second connecting section has the next smaller distance to the post. It is thus adjacent to the first connecting section and has a larger distance to the considered post than the first connecting section.

In some of the embodiments of the implant according to the invention, a third connecting section has a third thickness d3. Thickness d3 is larger than thickness d2. It is thus adjacent to the second connecting section; however, it is not adjacent to the first connecting section and has a larger distance to the considered post than the first connecting section and the second connecting section. In some of the embodiments of the implant according to the invention, a post is understood as a section of the supporting means which substantially is not curved, i.e., extends linearly.

In some embodiments, a connecting section is understood as a section of the connecting means which connects two or more bars with each other and which is more curved than at least one of the bars.

In some embodiments, a connecting section is understood as a section of the supporting means being a point of curvature in the sense of a curve progression. Thereby, the point of curvature of the connecting section is the point which is ever located most next to an end of the implant—with respect to the longitudinal direction thereof.

In some of the embodiments of the implant according to the invention, the implant is designed or embodied with cardiac valves—in particular artificial ones or ones which have been manufactured from animal tissue.

In certain embodiments according to the invention, bars comprised by the supporting means which connect a rod or post with the adjacent connecting sections are shorter than other bars. The shorter bars contribute to forming a slit that reaches from an end of the supporting means to the adjacent end of the post. The slit has a length of L2 (from its open end to its closed end).

From the opposite end of the slit to the adjacent end of string outlet or aperture 10 a distance having a length L3 is provided. Between the closed end of the slit and the centre of string outlet or aperture a distance having a length L4 is provided.

The open end of the slit may be spaced from the centre of the outlet or aperture by the sum of L2 and L4.

In a preferred embodiment, L1 is between 2.5 and 3.5 times as long as L2, preferably 3 times as long. L1 is the length of the supporting means in a distal-proximal direction thereof.

In some embodiments, L2 is 2 times (or between 1.5 and 2.5 times) the length of L4.

In certain embodiments, L2 is 3 times (or between 2.5 and 3.5 times) the length of L4.

A—preferably controlled—unfolding and/or re-folding of the implant (which can also include expanding and returning the implant to a reduced diameter) can be effected by means of a known catheter. The catheter can be designed as is described in US 2007/0100427 A1 by Perouse or in US 2005/0075731 A1 by Artof et al. By way of reference, the contents thereof are each subject-matter of the present invention. This particularly applies for the materials and (part) geometries given therein.

Regarding its basic design, the implant can be constructed as is, for example, described in DE 10 2008 013 948 A1 by the applicant of the present invention. By way of reference, the content thereof is also subject-matter of the present invention. This particularly applies for the materials and (part) geometries given therein.

The implant can be self-expanding, for example, it can be formed from or with a memory material, in particular nitinol, or materials which comprise nitinol. The implant can also be partly self-expanding, partly by the use of an expanding means. The implant can exclusively be non-self-expanding. The implant can be foldable or collapsible, respectively; the implant can be non-foldable or non-collapsible, respectively.

The implant can comprise a biocompatible material, in particular a biocompatible stainless steel. The material can be bio-absorbable.

The implant can be designed with or without a means for encompassing or sandwiching parts of native valve sections (in particular heart valve leaflets). In particular, the implant can be designed with or without sections rising up or lowering down due to temperature and memory effect.

The object according to the invention is also solved by the feature combination of the set according to claim 11 and by the feature combination of the supporting means according to claim 12.

According to the invention, a set is proposed which comprises at least one implant according to the invention, in particular a stent; and at least one catheter. Furthermore, there is proposed a supporting means which is provided or suited for use in an implant according to the present invention and which thus converts or can convert an implant not embodied according to the invention into an implant according to the present invention.

The advantages achievable herewith correspond to at least those of the implant according to the invention.

The following advantages may be found in certain embodiments according to the invention.

By using the implant according to the invention, an expansion thereof is, according to the invention, possible without distorting or warping, respectively, or undulating or waving, respectively, the supporting structure. In some embodiments according to the invention, this can primarily enable a multiple, however, at least a twofold, re-expansion following a happened reduction of the cross-section. The desired expansion behavior of the supporting means is maintained in such a case, too. A distortion or warping, respectively, or an undulation or waving, respectively, does not take place in such a case, too. The latter advantageously allows for a precise positioning of the implant. It can further ensure the more accurate residing of the supporting means and, thus, of the implant against the tissue of the implantation site.

Further, the likelihood of any undue or adverse stress concentration within the solid parts of the implant can advantageously be avoided or minimized.

The invention is exemplarily illustrated by means of the appended figures. In the figures, same or similar structures are denoted by the same reference numerals throughout the figures, wherein:

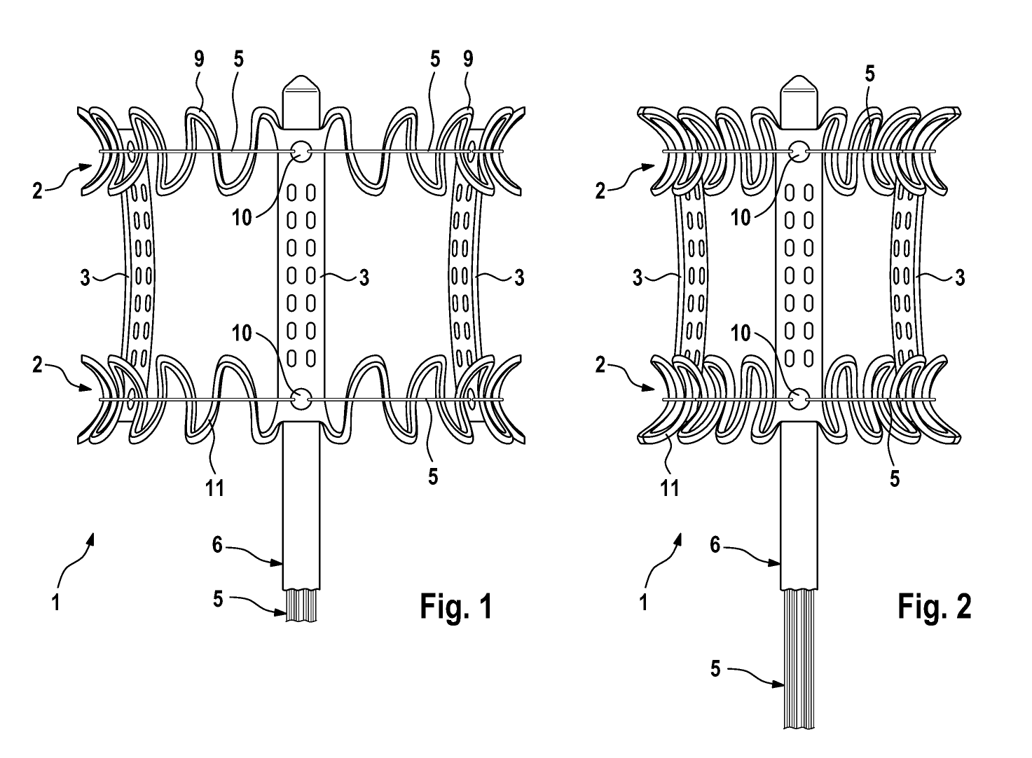

FIG. 1 shows a medical implant according to the present invention in an expanded state which is expandable and can be reduced in its diameter by use of a means;

FIG. 2 shows the medical implant of FIG. 1 in a non- or less expanded state;

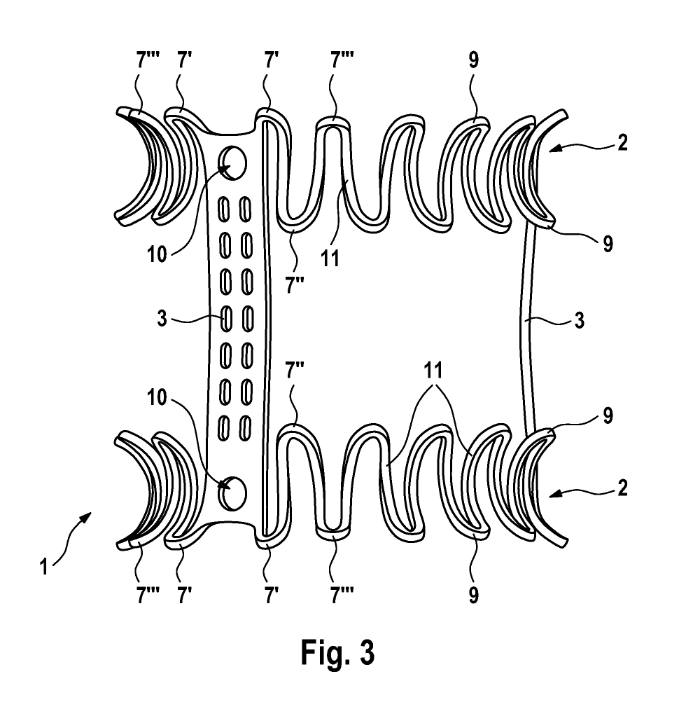

FIG. 3 shows enlarged sections of the medical implant of FIG. 1;

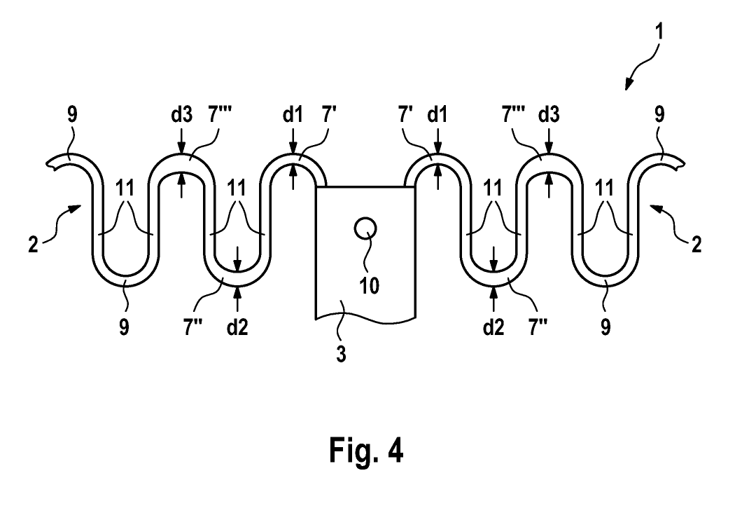

FIG. 4 shows enlarged sections of a supporting means of the implant of FIGS. 1 to 3 according to the invention; and

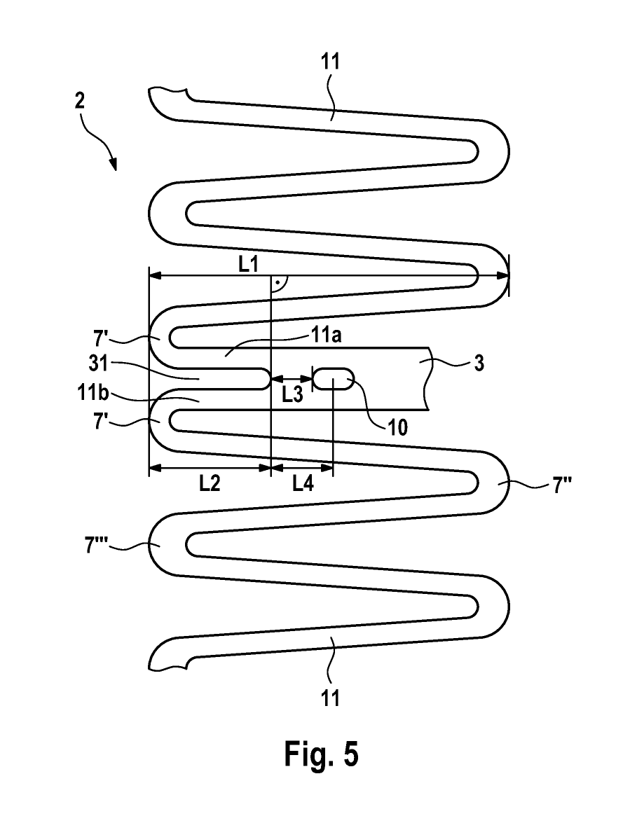

FIG. 5 shows an enlarged section of a supporting means of the implant according to the invention.

FIG. 1 shows an implant 1 which is expandable and can be reduced in its diameter. The diameter thereby refers to a plane perpendicular to a longitudinal axis of the medical implant 1. The longitudinal direction also corresponds to the direction of the extension of the catheter 6 shown in FIG. 1. The implant 1 comprises two circular supporting means or rings 2. The supporting means 2 are connected to rods or posts 3. In some embodiments, the supporting means 2 can—additionally or alternatively or exclusively—fulfill the function of a guiding means for reins 5. The reins 5 form part of a catheter 6 and serve for applying force or tension or stress, respectively, to the supporting means 2 for the purpose of expanding or folding the implant in a targeted manner. In the example of FIG. 1, the supporting means 2 are each designed in form of an outwardly half-open channel, through which the reins 5 are guided. The half-open channel is opened in a direction away from the center of the implant 1. However, the channel can also be designed in a form open to the implant or to another direction.

In the example of FIG. 1, the supporting means 2 are interrupted by posts 3, i.e. the posts 3 are integrated into the supporting means 2 such that they form sections of the supporting means 2.

In the embodiment of the implant 1 according to the invention of FIG. 1, the supporting means 2 have (round or differently shaped, e.g., oval, rectangular, elliptic, and so on) passage means or apertures 10. In the embodiment of the implant 1 according to the invention, these serve as a passage for the reins 5.

Furthermore, the implant 1 can also comprise a number of guiding means other than two, for example, one, three, four or more guiding means. The supporting means 2 can be arranged circularly, however, they can also be arranged non-circularly. The supporting means 2 can be formed integrally with the implant; however, they can also be fabricated separately. The supporting means 2 can have the form of a wave or undulation, respectively; however, they can also be fabricated in any other form, in particular, a non-wavy or non-undulating form.

Independent of all other features, the implant 1 can be fabricated from flat material, e.g., a material which has been cut with a laser, wherein, e.g., after having designed a pattern in the flat material, the material is reformed into a tube (optionally by connecting, such as welding, longitudinal sides of the former flat material lane or web, respectively). However, the implant 1 can also be fabricated from a tubular material directly.

The supporting means 2 of the implant 1 consist of a plurality of bars 11 which are each connected to another by means of connecting sections 9. According to the invention, the connecting sections 9 differ in their design. However, as the latter is not shown in FIG. 1, reference is made to FIG. 4.

FIG. 2 shows the implant 1 of FIG. 1. Two reins 5 have been led around the implant 1 and return back to the catheter 6 through the respectively same passage means or apertures 10. The reins 5 apply a tension or stress, respectively, on the implant 1 and the implant 1 is not completely expanded or unfolded, respectively. The diameter of the implant 1 has been reduced.

FIG. 3 shows an enlarged section of the medical implant of FIG. 1. In this enlargement, connecting sections 7′, 7″, 7′″ and 9 can be seen. All of them connect bars 11 which are arranged there between.

FIG. 4 shows a detail of a supporting means 2 of the implant 1 according to the invention. For illustration purpose, this detail of the supporting means 2 is shown as an even structure in FIG. 4.

Connecting means 7′ are shown which are each followed by a bar 11 and subsequent connecting sections 7″. Those are again followed by another bar 11 which is in turn followed by another connecting section 7′″. Respective bars 11 and, finally, connecting sections 9 follow the connecting sections 7′″ at both ends of the post 3.

FIG. 4 shows that the connecting sections T, 7″ and 7′″ each have widths d1, d2 and d3. Thereby, in the presentation of FIG. 4, width d1 is smaller than width d2 and width d2 is smaller than width d3. That means: d1<d2<d3.

In the embodiment according to the invention shown in FIG. 4, the connecting section is an apex of curvature or comprises such an apex of curvature.

The difference of the widths d1, d2 and d3 is present in a direction which extends in parallel to a longitudinal axis of the medical implant 1.

In other embodiments according to the invention of the supporting means according to the invention, the differences are present in another direction, e.g., in a direction which does not extend in parallel to a longitudinal axis of the medical implant during a state of use (e.g., before an extracorporeal expansion) or in turn in another direction. This other direction can be a direction perpendicular to a longitudinal axis of the medical implant 1. Moreover, this other direction can be any other direction.

The widths of the connecting sections 9 can, e.g., correspond to width d3. However, the connecting sections 9 can also have any other width. In particular, the connecting sections 9 can have a uniform width.

FIG. 5 shows an enlarged section of the supporting means 2 of the implant 1 according to one embodiment of the invention. In the example of FIG. 5, the supporting means has a width of L1 (from the distal end to the proximal end).

As can be seen from FIG. 5, the supporting means 2 comprises bars 11a and 11b which connect a rod or post 3 comprising an (oval) string outlet or aperture 10 with corresponding adjacent connecting sections 7′, respectively. Bars 11a and 11b which can be considered as to merge with the post 3 (in contrast to other bars 11) are shorter than other bars 11. In fact, bars 11a and 11b contribute to forming a slit 31 that reaches from an end of the supporting means 2 shown at the left-hand side of the representation of FIG. 5 to the left-hand end of rod or post 3. In FIG. 5, the slit has a length of L2.

As can further be derived from FIG. 5, between the right-hand end of slit 31 and the left-hand end of string outlet or aperture 10 a distance having a length L3 is provided. The distance L3 may be filled with a solid part of post 3.

Between the right-hand end of slit 31 and the centre of string outlet or aperture 10 a distance having a length L4 is provided.

The left-hand end of the supporting means 2 of FIG. 5 may be spaced from the centre of the outlet or aperture 10 by the sum of L2 and L4.

As regards to the relation of L1, L2, L3, and L4, it is noted that in one preferred embodiment of the invention, L1 is between 2.5 and 3.5 times as long as L2, preferably 3 times as long.

In some embodiments, L2 is 2 times (or between 1.5 and 2.5 times) the length of L4. In certain embodiments, L2 is 3 times (or between 2.5 and 3.5 times) the length of L3.

As regards FIG. 5, it is noted that the left-hand end of the supporting means 2 may be the distal or proximal end of the supporting means 2 and/or of the implant 1.

It is to be noted that the features described with reference to FIG. 5 may be embodied in an implant according to the invention without necessarily comprising also features described with regards to FIGS. 1 to 4.

1. An implant comprising:
at least one supporting arrangement which is suited for supporting the implant at or on an implantation site, wherein both the supporting arrangement and the implant are expandable from a first diameter to a second diameter and/or are collapsible from the second diameter to the first diameter, wherein the supporting arrangement comprises:
bars which are connected to each other, by means of connecting sections; and
at least one post for connecting the supporting arrangement with at least one other structure of the implant, wherein at least two of the connecting sections differ in at least one material or one geometrical characteristic.

2. An implant according to claim 1, wherein the material or the geometrical characteristic is a thickness of at least one section of the connecting sections.

3. An implant according to claim 1, wherein a first connecting section having the smallest distance to a post has a first thickness d1, wherein thickness d1 is the smallest thickness of all thicknesses of the connecting sections.

4. An implant according to claim 3, wherein a second connecting section having the next smaller distance to the post has a second thickness d2, wherein thickness d2 is larger than thickness d1.

5. An implant according to claim 3, wherein a third connecting section having in turn the next smaller distance to the post has a third thickness d3, wherein thickness d3 is larger than thickness d2.

6. An implant according to claim 1 which is designed or embodied with cardiac valves.

7. An implant (1) according to claim 1, comprising a shape memory material, in particular nitinol, or consisting thereof.

8. An implant comprising:
at least one supporting arrangement which is suited for supporting the implant of claim 1 at or on an implantation site, wherein both the supporting arrangement and the implant are expandable from a first diameter to a second diameter and/or are collapsible from the second diameter to the first diameter, wherein the supporting arrangement comprises bars which are connected to each other by means of connecting sections, the implant comprising
one or more posts interconnected to one or more supporting arrangements having bars, wherein two of those bars are merging with the post to form a slit having an open and a closed end, wherein a length of the slit fits between 2.5 and 3.5 times, in the length of the supporting arrangement in a distal-proximal direction thereof.

9. An implant according to claim 8, wherein a distance between the closed end of the slit and the neighbouring end of the adjacent aperture is one third of the length of the slit.

10. An implant according to claim 8, wherein a distance between the closed end of the slit and the centre of the adjacent aperture is half of the length of the slit.

11. A set comprising at least one expandable and/or decollapsible implant, according to claim 1; and at least one catheter or other delivery device for inserting the medical implant.

12. A supporting arrangement for designing or embodying an implant according to claim 1.
